(19) United States
(12) Patent Application Publication
(10) Pub. No.: US 2007/0256694 A1
(43) Pub. Date: Nov. 8, 2007

(54) System to alleviate snoring

(76) Inventor: Leon Kussick, Naples, FL (US)

Correspondence Address: Leon Kussick DDS, 3884 Clipper Cove Dr., Naples, FL, 34112, US

(21) Appl. No.: 11/418,506

(22) Filed: May 5, 2006

(51) Int. Cl. A61F 5/56

(52) U.S. Cl. 128/848

(57) ABSTRACT

The anti-snoring device is a combination of a chin pillow placed under the chin of a wearer and is designed to induce the wearer's mouth to stay closed while sleeping. Included in the combination is a dental appliance that has an upper box to receive the wearer's upper teeth therein. Below the box is an indent to receive the wearer's lower teeth therein to enhance the wearer's proper bite or occlusion when the mouth of the wearer is closed. Included in the appliance are two upper segments that extend under the roof or palate of the mouth of the wearer. From below the box are extending two inclines which follow a line adjacent the position of the lower teeth without touching them. The ultimate aim is to induce a person wearing the chin pillow or the dental appliance or both to keep his or her mouth closed when sleeping to thereby alleviate the incident of snoring.

### FIELD OF THE INVENTION

Snoring is a well known occurrence in many people due to the fact that the soft palate tissues in the airways are vibrating while sleeping causing the snoring. Snoring is an occurrence that is caused by the soft palate tissues vibrating of the tissues which vibration creates a sound like snoring which is usually caused by the person's mouth being open in a sleeping posture throughout the night. A major issue then is to keep the person's mouth closed during the night while sleeping to avoid breathing through the mouth.

### BACKGROUND OF THE INVENTION

There are many treatments known in the medical field to alleviate or prevent the above mentioned problem. One treatment is known as the pillar palatal implant stiffening soft palate system, it is widely used because it is cheaper to implement and far less painful than traditional snoring surgery. This system involves braids that are sewn or installed in the soft palate and/or uvula, which when vibrating makes much of the noise when breathing with mouth open, and therefore, alleviating the open mouth vibration of the soft palate and local tissues. The stiffening of the soft palate may help not to close off the airways which causes snoring. The pillar implant has the advantage that the inserts can be removed if they don't work or are not effective. Sleep apnea, that radically lowers blood oxygen and causes hypertension with the possibility of strokes and heart disease or arrhythmia of the heart beat. There are some 80 dental appliances being used which are not recommended because they impact on the lower mandible and may result in a long term deformation of the mandible which is not desirable at all when treating the problem of snoring. Then there is the use of the so-called CPAP mask which is sometimes called the “Gold Standard” in the treatment of snoring. However, this mask is difficult to wear and to use for many people because it is uncomfortable and causes claustrophobia in many people.

Of the many treatments for reducing snoring or apnea or both, none are routinely successful. Neither surgical or dental appliances, which usually force the mandible forward ½ or more of its maximum protrusion, are not recommended to treat the symptoms of snoring. This prevents routine safe improvements in long term help with frequent adjustments of occlusions or mold changes. Traditional operations that have been performed, involve cutting away the uvula and soft palate, removing tonsils, trimming the back of the tongue or sawing the mandible apart to move the mandible or tongue forward. All have the common goal to keep the airway into the trachea open at all times. Uvolopalatopharyngoplasty, known as “palate trimming”, requires hospitalization and often leaves the patient in pain for weeks and is only effective in about half the times.

Other procedures involve the bombardment of the soft palate with radio waves to stiffen the same or another procedure that involves putting a screw and ligature inside the chin to hold down the base of the tongue. Still other remedies, that are suggested, is the use of the CPAP mask that keeps the airways into the mouth closed but forces air into the patient to breathe through the nose. This treatment has not been routinely successful because many patients have claustrophobia and cannot tolerate the mask while sleeping.

### BRIEF DESCRIPTION OF THE INVENTION

The inventive concept involves the use of an appliance that is retained and worn in the upper half of the mouth close to the upper roof of the mouth palate. The appliance has an upper anterior retention box in which the upper front teeth fit tightly into that box and the appliance has two forward inclines or slants that do not touch the back of the lower teeth as the inclines guide the lower jaw to slide up to the top of the two inclines into a normal closure of the bite or occlusion of the snore patient. This way the lower mandible will not be distorted over a period of time to be effective as a snoring device. And for that purpose, as the teeth, occlusion or bite will come together as the back of the tongue is attached to the mandible, while it is attached to the jaw, will come forward, similar to a normal closed mouth position similar to the way we breathe, mouth closed but through the nose. Thereby, the soft tissue in the back of the tongue is pulled forward and is not resting against the back of the throat or uvula (soft palate) to enhance the breathing and to prevent any snoring. The object is to locate the jaw but do not force it forward. The mandible has to stay forward and with it the tongue. The mouth has to stay closed, that is, the upper and lower teeth should stay in contact with the appliance that is located between the upper and lower teeth. The inventive anti-snoring device or system includes a pad represented by a pillow that is placed under the front of the jaw and against the “Adams Apple” at a midway position and at the collar bone at its lower end of a person without asserting much pressure to create discomfort but it is effective enough to keep the mouth closed all night. The pad in the form of a pillow has straps thereon to be placed around the neck of the wearer so that it cannot be unintentionally dislodged while the person is sleeping. The straps have closure ends thereon in the form of a separable fasteners or other means. The tension of the straps can be adjusted so that the mouth of a patient may be kept closed all night through. This will prevent open mouth breathing so as not to cause vibrations of the soft tissue at the back of the throat.

### BRIEF DESCRIPTION OF THE DRAWINGS

FIGS. 1-3 illustrate a chin pillow or pad under the chin of a patient in various positions under the chin of a patient;

FIG. 4 illustrates the dental appliance as it is installed in a patients mouth;

FIG. 5 is a perspective view of the dental appliance.

### DETAILED DESCRIPTION OF THE INVENTION

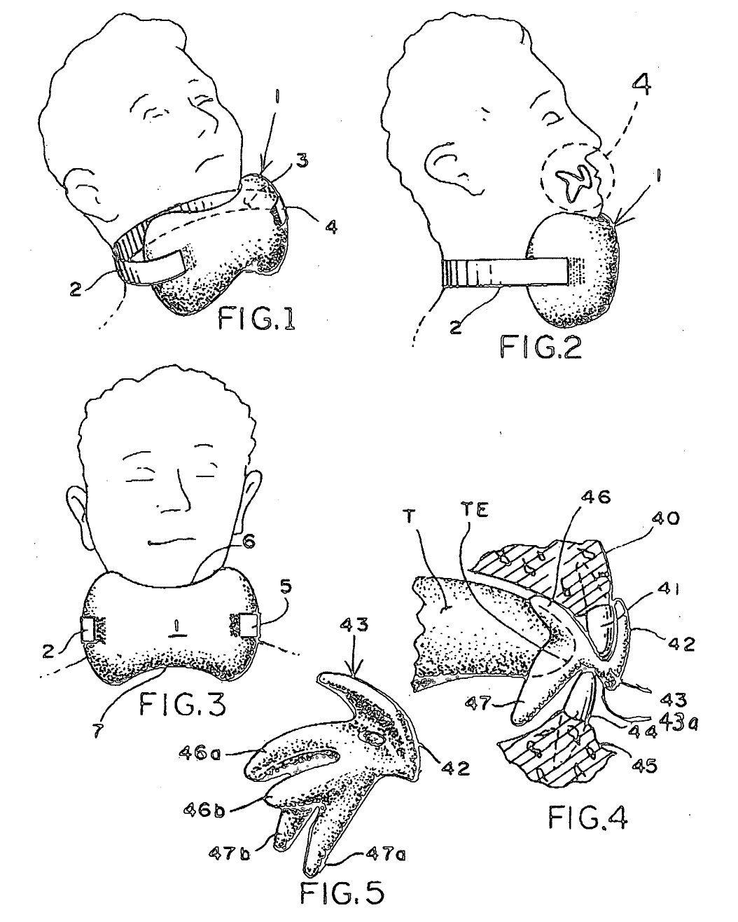

FIGS. 1-3 show the chin pillow 1 as it is worn under the chin of the patient. As was discussed above, the ultimate aim to prevent or at least alleviate snoring is that the patient will have to keep his/her mouth closed while sleeping so that the patient must breath through the nose. To this end the chin pillow or pad fits tightly under the chin and may embrace the same by way of a depression 6. The lower part of the pillow will find a support on the collar bones in the upper chest area. For this purpose, there is provided a curved section 7 which enhances the fit on the collar bones. The chin pillow or pad is held in place by way a strap which passes around the neck of the wearer and may be fastened to the pad 1 by adjustable fasteners such as hook and loop fasteners 4 and 5. This chin pillow or pad is comfortable to wear and experience has shown that it does not interfere with the patient's sleeping condition or habits. It may also be advisable to use an additional and somewhat wider strap around the patient's neck and pad independent of the pad itself to thereby aid in securing the pad in its adjusted position. Experience has also shown that, especially initially when trying to treat the condition of snoring a dental appliance should be used which assures that the teeth stay in a proper position when the patient is sleeping. By proper position is meant that the teeth remain closed and in their proper occlusion or bite alignment.

FIG. 4 illustrates such a dental appliance as it is installed in patient's mouth. The dental appliance has an upper box 43 to receive the front teeth 41 the box 43 is formed by an upwardly extending extension 42 which almost reaches the upper gum 40. Below the box 43 there is a depression 43 which will receive the lower teeth 44 located in the gum 45. The appliance also has two downwardly directed inclines 47a and 47b which are located behind the lower teeth 41. The inclines 47a and 47b should not exert any pressure on the lower teeth and thereby on the mandible because it would deform the lower mandible to a different state that may be useful in an orthodontist procedure which is not the aim of the present invention. As can be seen from FIG. 4, there are also two upper horizontal segments 46a and 46b which will seats themselves against the roof of the mouth as a stabilizing factor. In FIG. 4, the tongue T aligns itself against and within the dental appliance and an extension TE of the tongue T will settle itself within the appliance at a forward location. This, then results in that the mouth of the patient will stay closed during the sleeping time and the tongue will automatically stay in a forward position which will result in a non-snoring condition of a patient. This was explained above in that the soft tissue at the back of the tongue Fig. is a perspective view of the dental appliance which shows the upper two upper segments 46a and 46b which seat against the upper roof of the mouth as a stabilizing factor. FIG. 5 also shows the two lower inclines 47a and 47b which are located in the vicinity of the lower teeth 44 but not in contact therewith. Because of all of the above, it can be seen that the dental appliance will keep the mouth of the patient in a closed position to thereby prevent the soft tissue in the back of the throat from air passing there over because the air passing over the soft tissue is diverted into a different direction and, therefore, cannot cause any vibration of the soft tissue to thereby alleviate snoring.

What I claim is:

1. a combination device to eliminate snoring, said combination includes a: chin pillow placed under the chin of a patient, said chin pillow inducing said patient to keep his/her mouth closed, said combination further including a dental appliance placed between the upper and lower teeth, said appliance keeping said upper and lower teeth in a proper occlusion, whereby, said mouth of said patient will be kept in a closed position.

1. The device of claim 1, wherein said chin pillow is placed under the chin of said patient and at a counter point of the collar bones of the upper chest area of the patient.

2. The device of claim 1 including an adjustable strap placed around the neck of the patient and a forward surface of said chin pillow.

3. The device of claim 2, wherein said strap includes adjustable end pieces.

4. The device of claim 1, wherein said dental appliance includes an upper facing box into which upper teeth of the patient are located.

5. The device of claim 4, wherein said dental appliance further includes inclines located downwardly from said box and adjacent lower teeth of said patient.

6. The device of claim 1 including at least two upper segments extending from said dental appliance, said segments are placed against the upper roof or palate of the patient's mouth when worn.

7. The device of claim 6, wherein the tongue of said patient is placed against said appliance and under said at least segments to keep said tongue into a forward extended position.

8. A device to alleviate snoring consisting of a chin pillow placed under the chin of a wearer in combination with a dental appliance that is placed between the upper and lower teeth of said wearer, said chin pillow has a tendency to keep said wearer's mouth closed when sleeping, said dental appliance having a frontal box therein to receive said wearer's upper teeth therein, said appliance further having upper segments extending under the roof of the mouth of the wearer, said dental appliance further having inclines extending from below said box downwardly adjacent the lower teeth of said wearer without touching the lower teeth.
